(19) United States
(12) Patent Application Publication
(10) Pub. No.: US 2022/0354975 A1
(43) Pub. Date: Nov. 10, 2022

(54) Sterilizing Mouthpiece Assembly

(71) Applicant: Calden Mares, Temecula, CA (US)

(72) Inventor: Calden Mares, Temecula, CA (US)

(21) Appl. No.: 17/308,669

(22) Filed: May 5, 2021

(51) Int. Cl. A61L 2/00; A61L 2/26; A61N 5/06

(52) U.S. Cl. CPC A61L 2/0047; A61L 2/26; A61N 5/0624; A61L 2202/11; A61N 2005/0626; A61N 2005/0661; A61N 2005/0645

(57) ABSTRACT

A sterilizing mouthpiece assembly for reducing the airborne transmission of infectious diseases includes a mouthpiece for positioning in a user's mouth. A shelf is integrated into the mouthpiece and a wedge extends upwardly from the shelf. A plurality of first light emitters is each of the first light emitters is integrated into the mouthpiece and each of the first light emitters emitting light in the ultraviolet spectrum to sterilize the inside of the user's mouth. A plurality of second light emitters is each positioned on the wedge and each of the second light emitters emits light in the ultraviolet spectrum to sterilize the inside of the user's mouth.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sterilizing devices and more particularly pertains to a new sterilizing device for reducing the airborne transmission of infectious diseases. The device includes a mouthpiece that has a shelf and a wedge extending upwardly from the shelf. A plurality of light emitters, each emitting ultraviolet light, is integrated into the mouthpiece and the shelf. In this way the light emitters sterilize the interior of a user's mouth when the mouthpiece is in the user's mouth.

### (2) Description of Related Art Including Information Disclosed Under 37 CFR 1.97 and 1.98

The prior art relates to sterilizing devices including a plurality of mouthpieces that each have a tooth channel integrated therein for receiving a user's teeth and a plurality of light emitters that emit light into the tooth channel for whitening the user's teeth. The prior art also discloses a variety of mouthpieces that include light emitters for emitting light onto a user's teeth for whitening the user's teeth. In no instance does the

### BRIEF SUMMARY OF THE INVENTION

An embodiment of the disclosure meets the needs presented above by generally comprising a mouthpiece for positioning in a user's mouth. A shelf is integrated into the mouthpiece and a wedge extends upwardly from the shelf. A plurality of first light emitters is each of the first light emitters is integrated into the mouthpiece and each of the first light emitters emitting light in the ultraviolet spectrum to sterilize the inside of the user's mouth. A plurality of second light emitters is each positioned on the wedge and each of the second light emitters emits light in the ultraviolet spectrum to sterilize the inside of the user's mouth.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a sterilizing mouthpiece assembly according to an embodiment of the disclosure.

FIG. 2 is a bottom perspective view of an embodiment of the disclosure.

FIG. 3 is a front phantom view of an embodiment of the disclosure.

FIG. 4 is a left side phantom view of an embodiment of the disclosure.

FIG. 5 is a perspective in-use view of an embodiment of the disclosure.

### DETAILED DESCRIPTION OF THE INVENTION

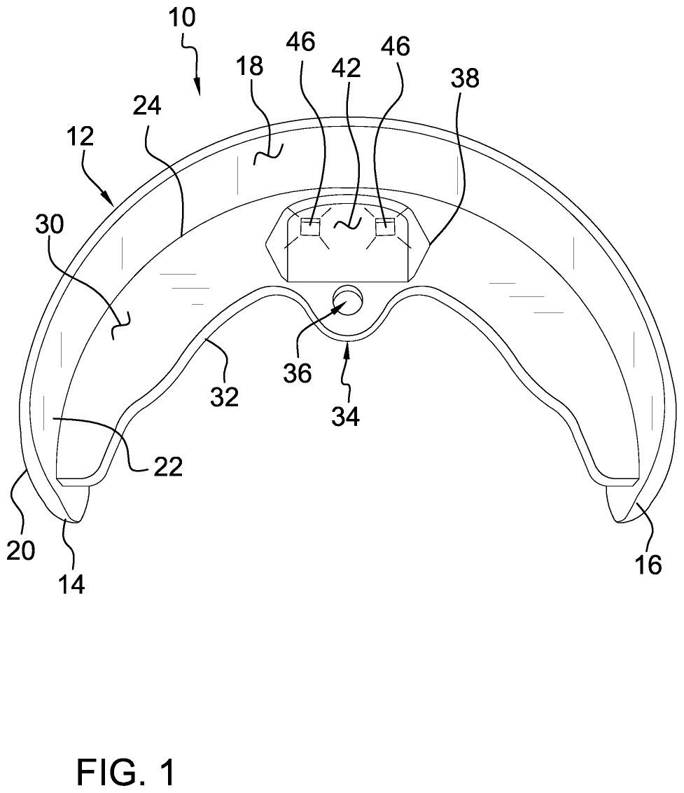

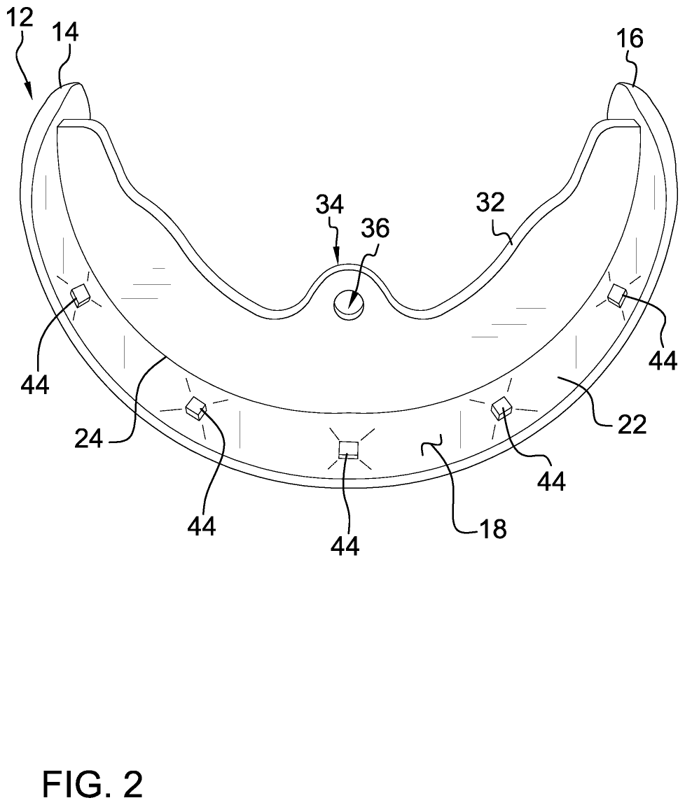

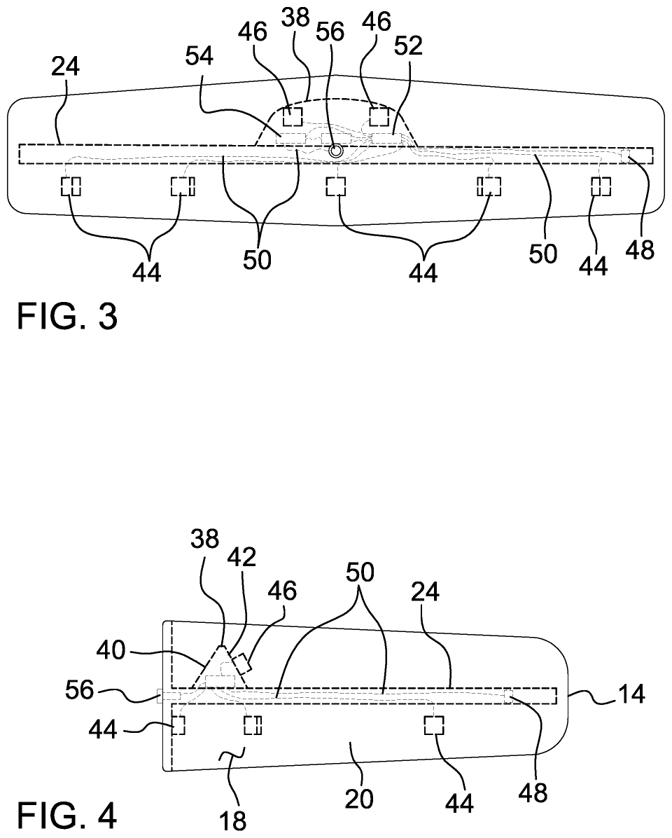

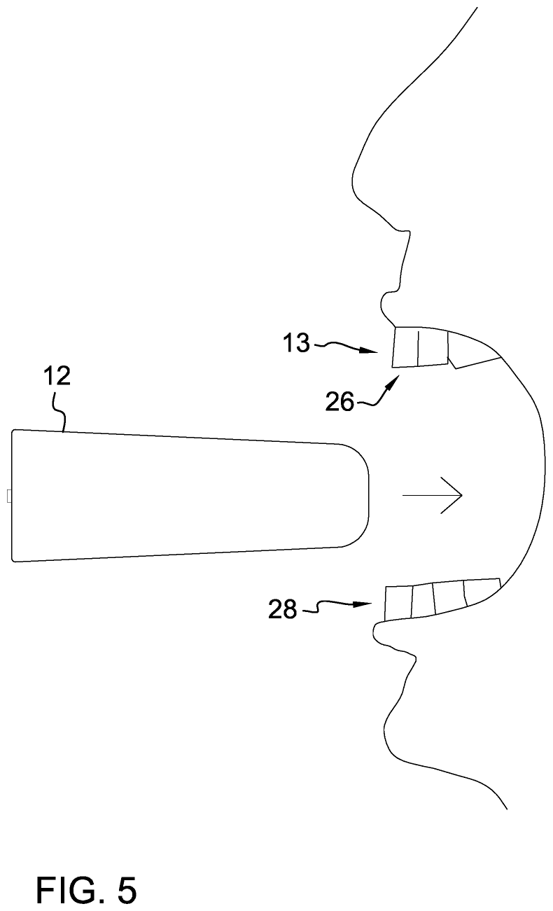

With reference now to the drawings, and in particular to FIGS. 1 through 5 thereof, a new sterilizing device embodying the principles and concepts of an embodiment of the disclosure and generally designated by the reference numeral 10 will be described.

As best illustrated in FIGS. 1 through 5, the sterilizing mouthpiece assembly 10 generally comprises a mouthpiece 12 that is comprised of a resiliently compressible material. In this way the mouthpiece 12 can be positioned in a user's mouth having the mouthpiece 12 being compressed between a user's teeth 13. The mouthpiece 12 has a first end 14, a second end 16 and an outer surface 18 extending therebetween, and the outer surface 18 has a front side 20 and a back side 22. The mouthpiece 12 is elongated between the first end 14 and the second end 16. Moreover, the mouthpiece 12 is curved between the first end 14 and the second end 16 having the first end 14 being spaced from the second end 16. In this way the mouthpiece 12 can conform to curvature of rows of the user's teeth 13.

A shelf 24 is integrated into the mouthpiece 12 such that the shelf 24 is positioned between an upper row of teeth 26 and a lower row of teeth 28. The shelf 24 is positioned on the back side 22, and the shelf 24 extends substantially between the first end 14 and the second end 16. The shelf 24 has a top surface 30 and a distal edge 32 with respect to the back side 22, and the top surface 30 lies on a plane that is oriented perpendicular to the back side 22. The distal edge 32 undulates along a full length of the distal edge 32 to define a lobe 34 that is centrally positioned on the shelf 24, and the lobe 34 is directed away from the back side 22. The shelf 24 has a hole 36 extending through the shelf 24 and the hole 36 is located on the lobe 34.

A wedge 38 is provided and the wedge 38 extends upwardly from the shelf 24. The wedge 38 is spaced from the mouthpiece 12 such that the wedge 38 is positioned behind the user's upper row of teeth 26 when the mouthpiece 12 is in the user's mouth. The wedge 38 is positioned on the top surface 30 of the shelf 24, and the wedge 38 has a front surface 40 and a back surface 42. The front surface 40 is spaced from the back side 22 of the outer surface 18 of the mouthpiece 12, and the back surface 42 is sloped.

A plurality of first light emitters 44 is each integrated into the mouthpiece 12 to emit light into the user's mouth when the mouthpiece 12 is in the user's mouth. Each of the first light emitters 44 emits light in the ultraviolet spectrum. In this way each of the first light emitters 44 can kill viruses and bacteria thereby sterilizing the inside of the user's mouth. Each of the first light emitters 44 is positioned on the back side 22 of the outer surface 18 of the mouthpiece 12. The first light emitters 44 are spaced apart from each other and are distributed between the first end 14 and the second end 16 of the mouthpiece 12, and each of the first light emitters 44 is positioned beneath the shelf 24. Each of the first light emitters 44 may comprise a light emitting diode or other type of electronic light emitter.

A plurality of second light emitters 46 is provided and each of the second light emitters 46 is positioned on the wedge 38. In this way each of the second light emitters 46 can light into the user's mouth when the mouthpiece 12 is in the user's mouth. Each of the second light emitters 46 emits light in the ultraviolet spectrum to kill viruses and bacteria thereby sterilizing the inside of the user's mouth. Additionally, each of the second light emitters 46 is positioned on the back surface 42 of the wedge 38. In this way the second light emitters 46 are angled upwardly in the user's mouth when the mouthpiece is in the user's mouth. Each of the second light emitters 46 may comprise a light emitting diode or other type of electronic light emitter.

A pressure switch 48 is integrated into the shelf 24 such that the pressure switch 48 can be actuated when the user bites down on the shelf 24. The pressure switch 48 is in communication with each of the first light emitters 44 and each of the second light emitters 46. Additionally, each of the first light emitters 44 and each of the second light emitters 46 are turned on when the pressure switch 48 is actuated. The pressure switch 48 is biased into a de-actuated condition for turning off each of the first light emitters 44 and each of the second light emitters 46.

A plurality of conductors 50 is each integrated into the mouthpiece 12 and each of the conductors 50 is electrically coupled to the pressure switch 48. Each of the conductors 50 is electrically coupled to a respective one of the first light emitters 44 or a respective one of the second light emitters 46. Additionally, each of the conductors 50 is comprised of an electrically conductive material.

A power supply 52 is integrated into the mouthpiece 12 and the power supply 52 is electrically coupled to the pressure switch 48. The power supply 52 comprises a rechargeable battery 54 that is integrated into the wedge 38 and the rechargeable battery 54 is electrically coupled to the pressure switch 48. A charge port 56 is recessed into the front side 20 of the outer surface 18 of the mouthpiece 12 to receive a charge cord. The charge port 56 is electrically coupled to the rechargeable battery 54 for charging the rechargeable battery 54.

In use, the mouthpiece 12 is inserted into the user's mouth and each of the first light emitters 44 and the second light emitters 46 are turned on when the user bites down on the shelf 24. In this way the first light emitters 44 and the second light emitters 46 sterilize the interior of the user's mouth. Thus, the user will be less likely to exhale viruses and bacteria that could be transmitted through the air. In this way the mouthpiece 12 can serve to reduce the airborne transmission of infectious diseases in a similar fashion as wearing a face mask.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sterilizing mouthpiece assembly for sterilizing the interior of a user's mouth, said assembly comprising:
a mouthpiece being comprised of a resiliently compressible material wherein said mouthpiece is configured to be positioned in a user's mouth having said mouthpiece being compressed between a user's teeth, said mouthpiece having a shelf being integrated into said mouthpiece wherein said shelf is configured to be positioned between an upper row of teeth and a lower row of teeth;
a wedge extending upwardly from said shelf, said wedge being spaced from said mouthpiece wherein said wedge is configured to be positioned behind the user's top row of teeth when said mouthpiece is in the user's mouth;
a plurality of first light emitters, each of said first light emitters being integrated into said mouthpiece wherein each of said first light emitters is configured to emit light into the user's mouth when said mouthpiece is in the user's mouth, each of said first light emitters emitting light in the ultraviolet spectrum wherein each of said first light emitters is configured to kill viruses and bacteria thereby sterilizing the inside of the user's mouth; and
a plurality of second light emitters, each of said second light emitters being positioned on said wedge wherein each of said second light emitters is configured to emit light into the user's mouth when said mouthpiece is in the user's mouth, each of said second light emitters emitting light in the ultraviolet spectrum wherein each of said second light emitters is configured to kill viruses and bacteria thereby sterilizing the inside of the user's mouth.

2. The assembly according to claim 1, wherein:
said mouthpiece has a first end, a second end and an outer surface extending therebetween, said outer surface having a front side and a back side, said mouthpiece being elongated between said first end and said second end, said mouthpiece being curved between said first end and said second end having said first end being spaced from said second end wherein said mouthpiece is configured to conform to curvature of rows of the user's teeth; and
said shelf is positioned on said back side, said shelf extending substantially between said first end and said second end, said shelf having a top surface and a distal edge with respect to said back side, said top surface lying on a plane being oriented perpendicular to said back side, said distal edge undulating along a full length of said distal edge to define a lobe being centrally positioned on said shelf, said lobe being directed away from said back side, said shelf having a hole extending through said shelf, said hole being located on said lobe.

3. The assembly according to claim 2, wherein said wedge is positioned on said top surface of said shelf, said wedge having a front surface and a back surface, said front surface being spaced from said back side of said outer surface of said mouthpiece, said back surface being sloped.

4. The assembly according to claim 2, wherein each of said first light emitters is positioned on said back side of said outer surface of said mouthpiece, said plurality of first light emitters being spaced apart from each other and being distributed between said first end and said second end of said mouthpiece, each of said first light emitters being positioned beneath said shelf.

5. The assembly according to claim 3, wherein each of said second light emitters is positioned on said back surface of said wedge.

6. The assembly according to claim 1, further comprising a pressure switch being integrated into said shelf wherein said pressure switch is configured to be actuated when the user bites down on said shelf, said pressure switch being in communication with each of said first light emitters and each of said second light emitters, each of said first light emitters and each of said second light emitters being turned on when said pressure switch is actuated, said pressure switch being biased into a de-actuated condition for turning off each of said first light emitters and each of said second light emitters.

7. The assembly according to claim 6, further comprising a plurality of conductors, each of said conductors being integrated into said mouthpiece, each of said conductors being electrically coupled to said pressure switch, each of said conductors being electrically coupled to a respective one of said first light emitters or a respective one of said second light emitters, each of said conductors being comprised of an electrically conductive material.

8. The assembly according to claim 6, further comprising a power supply being integrated into said mouthpiece, said power supply being electrically coupled to said pressure switch, said power supply comprising:
a rechargeable battery being integrated into said wedge, said rechargeable battery being electrically coupled to said pressure switch; and
a charge port being recessed into said front side of said outer surface of said mouthpiece wherein said charge port is configured to receive a charge cord, said charge port being electrically coupled to said rechargeable battery for charging said rechargeable battery.

9. A sterilizing mouthpiece assembly for sterilizing the interior of a user's mouth, said assembly comprising:
a mouthpiece being comprised of a resiliently compressible material wherein said mouthpiece is configured to be positioned in a user's mouth having said mouthpiece being compressed between a user's teeth, said mouthpiece having a first end, a second end and an outer surface extending therebetween, said outer surface having a front side and a back side, said mouthpiece being elongated between said first end and said second end, said mouthpiece being curved between said first end and said second end having said first end being spaced from said second end wherein said mouthpiece is configured to conform to curvature of rows of the user's teeth, said mouthpiece having a shelf being integrated into said mouthpiece wherein said shelf is configured to be positioned between an upper row of teeth and a lower row of teeth, said shelf being positioned on said back side, said shelf extending substantially between said first end and said second end, said shelf having a top surface and a distal edge with respect to said back side, said top surface lying on a plane being oriented perpendicular to said back side, said distal edge undulating along a full length of said distal edge to define a lobe being centrally positioned on said shelf, said lobe being directed away from said back side, said shelf having a hole extending through said shelf, said hole being located on said lobe;
a wedge extending upwardly from said shelf, said wedge being spaced from said mouthpiece wherein said wedge is configured to be positioned behind the user's top row of teeth when said mouthpiece is in the user's mouth, said wedge being positioned on said top surface of said shelf, said wedge having a front surface and a back surface, said front surface being spaced from said back side of said outer surface of said mouthpiece, said back surface being sloped;
a plurality of first light emitters, each of said first light emitters being integrated into said mouthpiece wherein each of said first light emitters is configured to emit light into the user's mouth when said mouthpiece is in the user's mouth, each of said first light emitters emitting light in the ultraviolet spectrum wherein each of said first light emitters is configured to kill viruses and bacteria thereby sterilizing the inside of the user's mouth, each of said first light emitters being positioned on said back side of said outer surface of said mouthpiece, said plurality of first light emitters being spaced apart from each other and being distributed between said first end and said second end of said mouthpiece, each of said first light emitters being positioned beneath said shelf;
a plurality of second light emitters, each of said second light emitters being positioned on said wedge wherein each of said second light emitters is configured to emit light into the user's mouth when said mouthpiece is in the user's mouth, each of said second light emitters emitting light in the ultraviolet spectrum wherein each of said second light emitters is configured to kill viruses and bacteria thereby sterilizing the inside of the user's mouth, each of said second light emitters being positioned on said back surface of said wedge;
a pressure switch being integrated into said shelf wherein said pressure switch is configured to be actuated when the user bites down on said shelf, said pressure switch being in communication with each of said first light emitters and each of said second light emitters, each of said first light emitters and each of said second light emitters being turned on when said pressure switch is actuated, said pressure switch being biased into a de-actuated condition for turning off each of said first light emitters and each of said second light emitters;
a plurality of conductors, each of said conductors being integrated into said mouthpiece, each of said conductors being electrically coupled to said pressure switch, each of said conductors being electrically coupled to a respective one of said first light emitters or a respective one of said second light emitters, each of said conductors being comprised of an electrically conductive material; and
a power supply being integrated into said mouthpiece, said power supply being electrically coupled to said pressure switch, said power supply comprising:
a rechargeable battery being integrated into said wedge, said rechargeable battery being electrically coupled to said pressure switch; and
a charge port being recessed into said front side of said outer surface of said mouthpiece wherein said charge port is configured to receive a charge cord, said charge port being electrically coupled to said rechargeable battery for charging said rechargeable battery.
